(19) United States
(12) Patent Application Publication
(10) Pub. No.: US 2025/0009418 A1
(43) Pub. Date: Jan. 9, 2025

(54) CATHETER

(71) Applicant: Japan Lifeline Co.,Ltd., Tokyo (JP)

(72) Inventors: Kohei SAKAKI, Tokyo (JP); Takafumi WATANABE, Tokyo (JP)

(21) Appl. No.: 18/252,810

(22) PCT Filed: Jan. 29, 2021
(86) PCT No.: PCT/JP2021/003329

§ 371 Date: Dec. 11, 2023

(51) Int. Cl. A61B 18/14

(52) U.S. Cl. CPC A61B 18/1492; A61B 2018/00267; A61B 2018/00577; A61B 2018/00595; A61B 2018/00946; A61M 25/0097

(57) ABSTRACT

To provide a catheter capable of improving convenience. A catheter (electrode catheter 1) according to an embodiment of the present invention includes a catheter shaft 11 extending along an axial direction (Z-axis direction) and having a near-distal end structure 6 including a plurality of electrodes 111, and a handle 12 mounted on a proximal end side of the catheter shaft 11. The handle 12 includes a handle body 121 extending along the axial direction and a slide mechanism 123 configured to be slidable along the axial direction in the handle body 121 and operated during a deformation operation in which the shape of the near-distal end structure 6 is changed between a first shape and a second shape. The first shape is a non-deployed shape in which the near-distal end structure 6 is not deployed along the axial direction, and the second shape is a deployed shape in which the near-distal end structure 6 is deployed from the non-deployed shape along the axial direction.

### CROSS REFERENCE TO RELATED APPLICATIONS

This application claims priority to International Application No. PCT/JP2021/003329, filed on Jan. 29, 2021, the entire contents of which are hereby incorporated by reference.

### TECHNICAL FIELD

The disclosure relates to a catheter having a catheter shaft.

### BACKGROUND

An example of a medical device having an electrode near its distal end includes a catheter (electrode catheter) in which such an electrode is provided at a catheter shaft (for example, Japanese Patent No. 3830521). In the catheter in Japanese Patent No. 3830521, the structure near the distal end of the catheter shaft is configured to be deformable.

### SUMMARY

In the above-described catheter, it is commonly desired to improve the convenience. It is desirable to provide a catheter capable of improving convenience.

A catheter according to an embodiment of the disclosure includes a catheter shaft extending along an axial direction and having a near-distal end structure including a plurality of electrodes, and a handle mounted on a proximal end side of the catheter shaft. The handle includes a handle body extending along the axial direction, and a slide mechanism configured to be slidable along the axial direction in the handle body and operated during a deformation operation in which a shape of the near-distal end structure is changed between a first shape and a second shape. The first shape is a non-deployed shape in which the near-distal end structure is not deployed along the axial direction, and the second shape is a deployed shape in which the near-distal end structure is deployed from the non-deployed shape along the axial direction.

In the catheter according to an embodiment of the disclosure, since the slide mechanism slidable along the axial direction is provided in the handle body, the deformation operation in which the shape of the near-distal end structure including the plurality of electrodes is changed between the first shape (the non-deployed shape) and the second shape (the deployed shape) is performed as follows. That is, in a state where the operator holds the handle body with one hand, the operator can perform the deformation operation on the slide mechanism with the one hand (the same hand). That is, for example, an operation with both hands of the operator as in the case of an operation of pushing a wire (deformation wire) used for the deformation operation into the handle body with the other hand is not required, and the deformation operation can be easily performed using only one hand of the operator.

Here, the near-distal end structure may be settable to any intermediate shape between the non-deployed shape and the deployed shape according to a slide position of the slide mechanism in the handle body. In this case, the near-distal end structure can be set to any intermediate shape, and thus the convenience can be further improved.

A distal end side of a deformation wire used in the deformation operation may be fixed to the near-distal end structure, and a proximal end side of the deformation wire may be fixed by the slide mechanism in a state of being inserted into a rigid tube in the handle body. In this case, the proximal end side of the deformation wire is inserted into the rigid tube in the handle body, thereby avoiding bending of the deformation wire in the handle body when the deformation operation is performed. As a result, the deformation operation is smoothly performed, and the convenience can be further improved.

In this case, the near-distal end structure may be configured such that an irrigation liquid is ejected to the outside. A distal end-side region of the rigid tube in the handle body may be configured to be insertable into a protective tube, so that the liquid flows into a gap between the protective tube and the rigid tube toward the near-distal end structure, and a liquid stop member configured to suppress leakage of the liquid may be provided in the handle body on a proximal end side of an inflow position of the liquid with respect to the gap. In this case, the irrigation liquid can be supplied toward the near-distal end structure using the gap between the protective tube and the rigid tube, and the leakage of the irrigation liquid is suppressed by providing the liquid stop member. As a result, the size of the handle body can be reduced, and the reliability of the handle as a product can be improved.

Further, the handle may further include a rotation operation portion that is rotated during a deflection action of deflecting a portion near the distal end of the catheter shaft. The rotation operation portion may include a rotating plate configured to be rotatable about a rotation axis perpendicular to the axial direction with respect to the handle body, the rotating plate being a portion operated during the rotation operation, and a path defining member formed using a member different from a member of the rotating plate and configured to define a non-rotational path through which the deformation wire used in the deformation operation passes. In this case, since the rotating plate operated during the rotation operation and the path defining member for defining the non-rotational path in the deformation wire used during the deformation operation are formed using different members, the non-rotational path of the deformation wire is maintained during the rotation operation. As a result, the rotation operation and the deformation operation can be easily performed with a simple structure, and the convenience is further improved.

In addition, the slide mechanism includes a knob which is a portion operated during the deformation operation, and the knob is disposed on both a front surface side and a back surface side of the handle body. In this case, when the slide mechanism slides along the axial direction, the knobs slide on both the front surface side and the back surface side of the handle body, and thus during the deformation operation, the rotational movements from both knobs in the intermediate direction cancel each other out. This avoids the possibility of loss of the operating load during the deformation operation, and as a result, the deformation operation is performed smoothly, and the convenience is further improved.

The near-distal end structure may include a branch point of the catheter shaft, a joining point located near the most distal end of the catheter shaft, and a plurality of branch structures each including the electrode and individually connecting the branch point and the joining point in a curved shape. The non-deployed shape is a petal shape formed by the plurality of branch structures, and the deployed shape is a shape in which the petal shape is deployed along the axial direction.

According to the catheter of an embodiment of the disclosure, the slide mechanism is provided in the handle body, such that the deformation operation for changing the shape of the near-distal end structure can be easily performed using only one hand of the operator. This makes it possible to improve the convenience when using the catheter.

### BRIEF DESCRIPTION OF DRAWINGS

FIGS. 1A and 1B are schematic diagrams illustrating a schematic configuration example of a catheter according to an embodiment of the disclosure.

FIG. 2 is an exploded perspective view illustrating a schematic configuration example of a handle illustrated in FIGS. 1A and 1B.

FIG. 3 is a plan view schematically illustrating a configuration example of a portion of the handle illustrated in FIG. 2.

FIG. 4 is a cross-sectional view illustrating a specific configuration example of a portion of the handle illustrated in FIGS. 1A and 1B.

FIG. 5 is a cross-sectional view illustrating a specific configuration example of another portion of the handle illustrated in FIGS. 1A and 1B.

FIGS. 6A to 6C are schematic diagrams illustrating an example of a deformed state near a distal end of a catheter shaft illustrated in FIGS. 1A and 1B.

FIGS. 7A to 7C are schematic diagrams illustrating an example of another deformed state near the distal end of the catheter shaft illustrated in FIGS. 1A and 1B.

FIG. 8 is a schematic plan view illustrating a schematic configuration of a catheter according to a comparative example.

FIGS. 9A and 9B are schematic cross-sectional views for explaining a function of a knob of the handle illustrated in FIG. 4.

### DESCRIPTION OF EMBODIMENTS

Embodiments of the disclosure will be described below in detail with reference to the drawings. Note that the description will be given in the following order.
1. Embodiment (Application Example of Slide Mechanism for Deformation Operation Near Distal End of Catheter Shaft)
2. Modified examples

### 1. Embodiment

### A. Schematic Configuration

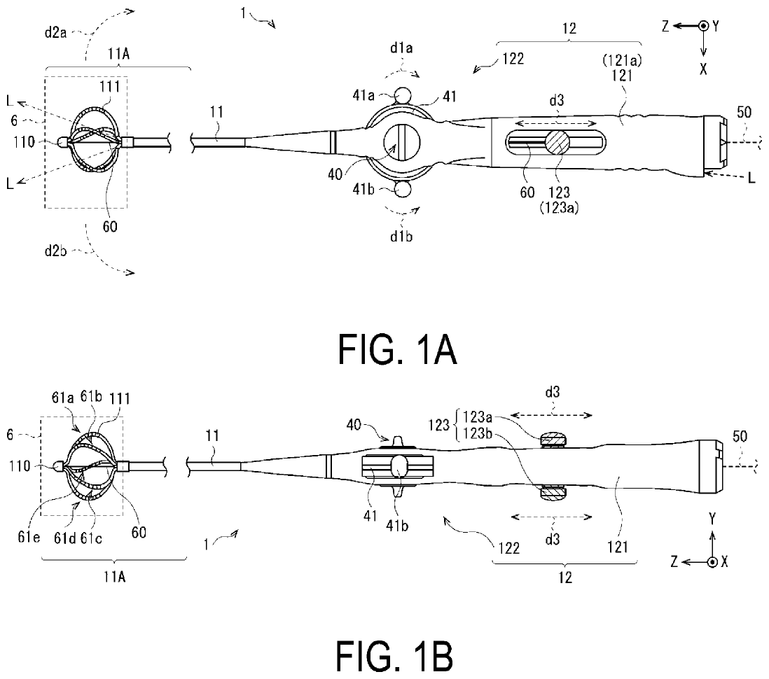

FIGS. 1A and 1B schematically illustrates a schematic configuration example of a catheter (electrode catheter 1) according to an embodiment of the disclosure. Specifically, FIG. 1A schematically illustrates a planar configuration example (Z-X plane configuration example) of the electrode catheter 1, and FIG. 1B schematically illustrates a side configuration example (Y-Z side configuration example) of the electrode catheter 1.

The electrode catheter 1 corresponds to a specific example of a “catheter” in the disclosure.

The electrode catheter 1 is a catheter that is inserted into a patient's body (for example, inside the heart) through a blood vessel and used for examination and treatment of arrhythmia or the like. Specifically, using a plurality of electrodes (electrodes 111), which will be described later, in the electrode catheter 1, measurement of electric potential near an affected area in the body, cauterization (ablation) of the affected area, and the like are performed.

Further, as illustrated in FIGS. 1A and 1B, the electrode catheter 1 includes an irrigation mechanism that supplies (ejects) a predetermined irrigation liquid L (for example, saline) to the outside from the vicinity of a distal end (a near-distal end structure 6 described later) during such ablation.

As illustrated in FIGS. 1A and 1B, the electrode catheter 1 described above includes a catheter shaft 11 (catheter tube) as a catheter body (elongated portion) and a handle 12 mounted on a proximal end side of the catheter shaft 11.

The handle 12 corresponds to a specific example of a “handle” in the disclosure.

### Catheter Shaft 11

The catheter shaft 11 has a flexible tube-like structure (made of a hollow tube-like member) and has a shape that extends along its axial direction (Z-axis direction) (see FIGS. 1A and 1B). Specifically, the length of the catheter shaft 11 in the axial direction is from several to several tens of times longer than the length of the handle 12 in the axial direction (Z-axis direction).

As illustrated in FIGS. 1A and 1B, the catheter shaft 11 includes a distal end portion (distal end flexible portion 11A) that is configured to have excellent relative flexibility. Further, as illustrated in FIGS. 1A and 1B, a predetermined near-distal end structure 6, which will be described later, is provided in the distal end flexible portion 11A. The catheter shaft 11 also has a so-called multi-lumen structure in which a plurality of lumens (inner holes, channels, through holes) are formed inside the catheter shaft 11 so as to extend along its axial direction (Z-axis direction). Various fine wires (lead wires 50, deflection wires, deformation wires 60, and the like, which will be described later) are inserted through the lumen of the catheter shaft 11 in a state of being electrically insulated from each other. Further, inside the catheter shaft 11, in addition to the lumens for inserting such various fine wires, a lumen for supplying the above-described irrigation liquid L is formed so as to extend along the axial direction.

The outer diameter of the catheter shaft 11 described above, for example, from approximately 1.0 to 4.0 mm, and the length of the catheter shaft 11 in the axial direction is, for example, from approximately 300 to 1500 mm. Examples of the constituent material of the catheter shaft 11 include a thermoplastic resin, such as polyamide, polyether polyamide, polyurethane, polyether block amide (PEBAX (trade name)), and nylon.

Here, as illustrated in FIGS. 1A and 1B, the near-distal end structure 6 includes a branch point (a position at the proximal end side of the near-distal end structure 6) of the catheter shaft 11, a joining point positioned close to the most distal end (close to a distal end tip 110 described later) of the catheter shaft 11, and a plurality (five in this example) of branch structures 61a to 61e that are portions to individually connect between the branch point and the joining point in a curved shape. These branch structures 61a to 61e are spaced apart from each other at substantially equal intervals in a plane (X-Y plane) perpendicular to the axial direction (Z-axis direction) of the catheter shaft 11.

Further, as illustrated in FIGS. 1A and 1B, these branch structures 61a to 61e have one or a plurality of electrodes 111 (four electrodes 111 in this example) spaced apart from each other at predetermined intervals along their curved extension directions. Each of the electrodes 111 is a ring-shaped electrode. On the other hand, the distal end tip 110 is arranged at the joining point of the branch structures 61a to 61e (near the most distal end of the catheter shaft 11).

As described above, the electrodes 111 described above are. for example, electrodes for potential measurement or cauterization, and are made of a metal material with good electrical conductivity such as, for example, aluminum (Al), copper (Cu), SUS, gold (Au), platinum (Pt), or the like. On the other hand, the distal end tip 110, as well as being constituted by a metal material similar to that of the electrodes 111, is constituted by a resin material such as a silicone rubber resin or a polyurethane.

The electrodes 111 described above are individually electrically connected to the distal end sides of the lead wires 50. Further, the proximal end side of each lead wire 50 can be connected to the outside of the electrode catheter 1 from the inside of the handle 12 through the inside of the catheter shaft 11. Specifically, as illustrated in FIGS. 1A and 1B, the proximal end side of each lead wire 50 is taken out to the outside from the proximal end portion (connector portion) of the handle 12 along the Z-axis direction.

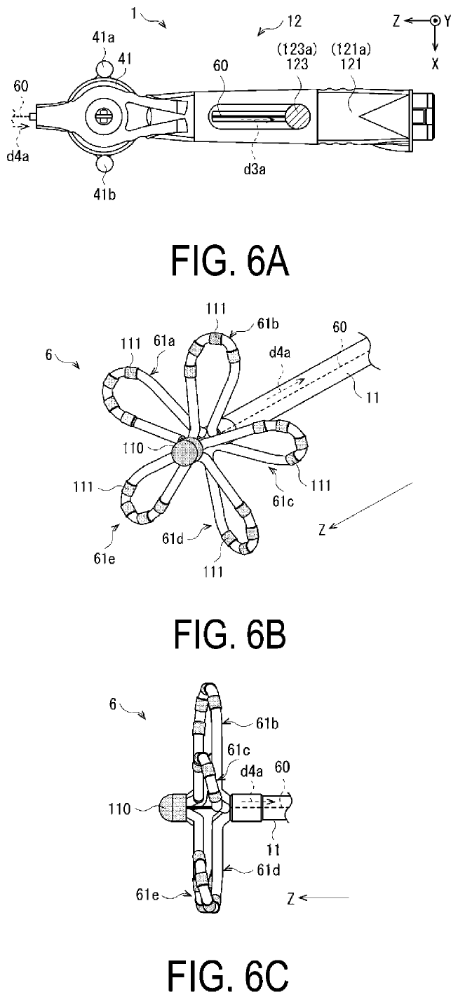

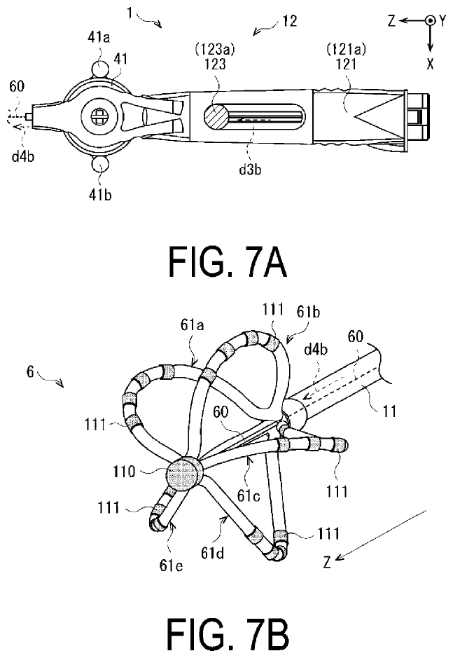

Here, such a shape of the near-distal end structure 6 is configured to change (deform) according to a deformation operation to be described later on the handle 12 (deformation operation to a slide mechanism 123 described later). Specifically, the shape of the near-distal end structure 6 changes between a non-deployed shape (contracted shape: see FIGS. 6A to 6C described later) in which the near-distal end structure 6 is not deployed along the axial direction (Z-axis direction) and a deployed shape in which the near-distal end structure 6 is deployed from the non-deployed shape along the axial direction (expanded shape: see FIGS. 1A and 1B and FIGS. 7A to 7C described later). Although the details will be described later, an example of such a non-deployed shape is a “petal shape” (an example of a flat shape: see FIGS. 6A to 6C described later) formed by the plurality of branch structures 61a to 61e. On the other hand, an example of the deployed shape is a shape in which such a petal shape (the branch structures 61a to 61e) is deployed along the axial direction (so-called “basket shape”: see FIGS. 1A and 1B and FIGS. 7A to 7C described later).

Note that the “basket shape” means that, for example, as illustrated in FIGS. 1A and 1B and FIGS. 7A to 7C, the shape formed by the plurality of branch structures 61a to 61e is a shape similar to a pattern of the curved shape formed on the surface of the basketball.

The non-deployed shape (and the petal shape) corresponds to a specific example of a “first shape” in the disclosure. Further, the deployed shape (and the basket shape) corresponds to a specific example of a “second shape” in the disclosure.

### Handle 12

The handle 12 is a portion that an operator (physician) grips (holds) when using the electrode catheter 1. As illustrated in FIGS. 1A and 1B, the handle 12 has a handle body 121 mounted on the proximal end side of the catheter shaft 11 and a rotation operation portion 122.

The handle body 121 corresponds to a portion (gripping portion) that an operator actually grips, and has a shape extending along its axial direction (Z-axis direction). The handle body 121 is made of a synthetic resin such as, for example, polycarbonate or acrylonitrile-butadiene-styrene copolymer (ABS).

Although the details will be described later, the rotation operation portion 122 is a portion that is operated during a deflection action for deflecting (bending) the vicinity (distal end flexible portion 11A) of the distal end of the catheter shaft 11 in two directions. The rotation operation portion 122 is used during such a deflection action together with a pair of deflection wires (not illustrated). Specifically, during such a deflection action, the rotation operation portion 122 is operated (rotated) by the operator of the electrode catheter 1. As illustrated in FIGS. 1A and 1B, the rotation operation portion 122 described above includes a lock mechanism 40 and a rotating plate 41.

The distal ends of the pair of deflection wires are fixed to the distal end side of the catheter shaft 11 (for example, near the distal end tip 110). The proximal end sides of the pair of deflection wires extend from the inside of the catheter shaft 11 to the inside of the handle 12 (the inside of the handle body 121).

As illustrated in FIGS. 1A and 1B, the rotating plate 41 is a member mounted on the handle body 121 so as to be rotatable with respect to the handle body 121 about a rotation axis (Y-axis direction: rotation axis Yr described later) perpendicular to its axial direction (Z-axis direction). The rotating plate 41 corresponds to a portion that is actually operated by the operator during the rotation operation, and has a substantially disk-like shape. Specifically, in this example, as indicated by arrows d1a and d1b in FIG. 1A, it is possible to perform an operation (rotation operation about the rotation axis Yr) of rotating the rotating plate 41 in two directions within the Z-X plane with respect to the handle body 121.

The lock mechanism 40 is a mechanism for fixing (locking) the rotational position of the rotating plate 41 within the Z-Y plane.

Here, as illustrated in FIGS. 1A and 1B, a pair of knobs 41a and 41b are provided integrally with the rotating plate 41 on the side surface of the rotating plate 41. In this example, as illustrated in FIGS. 1A and 1B, the knobs 41a and 41b are arranged at positions that are point-symmetrical about the rotation axis of the rotating plate 41. Each of these knobs 41a and 41b corresponds to a portion that is operated (pushed) by the fingers of one hand, for example, when the operator rotates the rotating plate 41. Note that the rotating plate 41 described above is constituted by a material (synthetic resin or the like) similar to that of the handle body 121 described above, for example.

A pair of fasteners (not illustrated) are provided on the rotating plate 41. These fasteners are members (wire fasteners) for individually fixing the proximal ends of the pair of deflection wires by screwing or the like. Note that, with these fasteners, it is possible to arbitrarily adjust the retraction length near each proximal end when fixing the proximal ends of the pair of deflection wires.

### B. Detailed Configuration of Handle 12

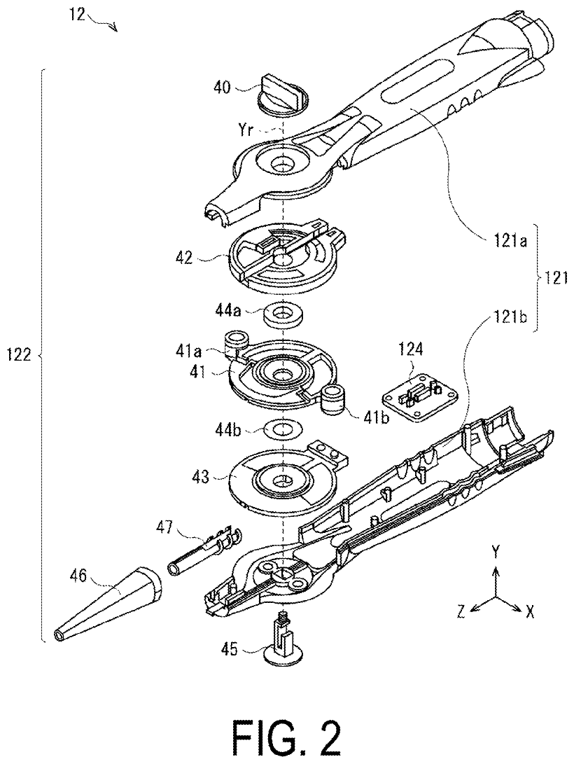

Next, with reference to FIGS. 2 to 5 in addition to FIGS. 1A and 1B, a specific configuration example of the handle 12 will be described.

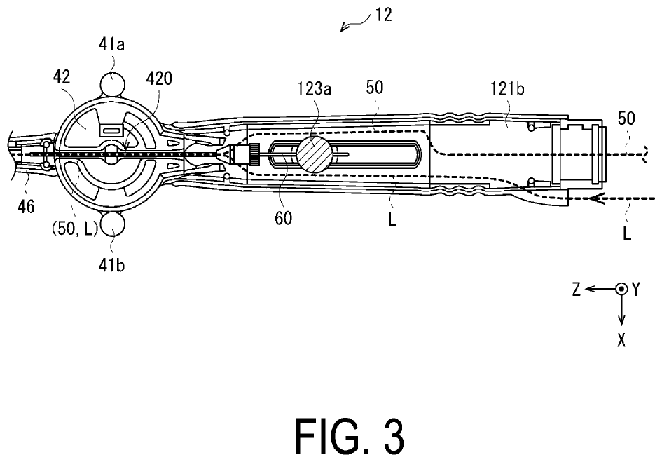

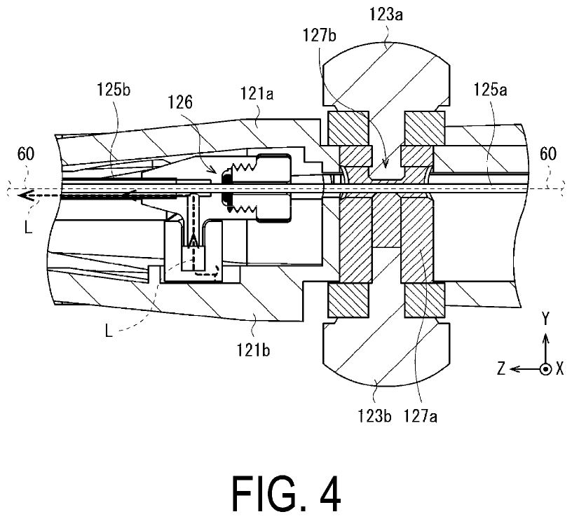

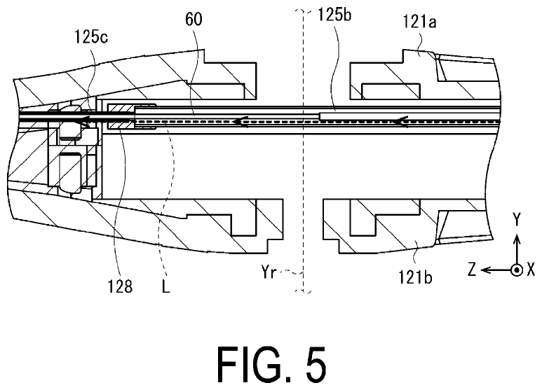

FIG. 2 is an exploded perspective view illustrating a schematic configuration example of the handle 12 (the slide mechanism 123 and the like, which will be described later, are omitted). FIG. 3 is a schematic plan view (Z-Y plan view) illustrating a configuration example of a portion of the handle 12 illustrated in FIG. 2 (a configuration example with a handle member 121a, described later, removed). FIG. 4 is a cross-sectional view (Y-Z cross-sectional view) illustrating a specific configuration example of a portion of the handle 12 (a portion near the slide mechanism 123, which will be described later). FIG. 5 is a cross-sectional view (Y-Z cross-sectional view) illustrating a specific configuration example of another portion of the handle 12 (a portion near the rotation axis Yr illustrated in FIG. 2).

First, as illustrated in FIG. 2, the handle body 121 is formed using a pair of handle members 121a and 121b that can be separated along the Y-axis direction. In other words, the handle body 121 is formed by connecting these handle members 121a and 121b to each other. As illustrated in FIG. 2, a fixing member 124 for fixing each path of the lead wire 50 and the deformation wire 60 in the handle body 121 is arranged in the handle member 121b.

Further, as illustrated in FIG. 2, the rotation operation portion 122 includes the lock mechanism 40 and the rotating plate 41, a path defining member 42, a rotating member 43, ring-shaped members 44a and 44b, and a connecting member 45, which are arranged along the rotation axis Yr in the Y-axis direction. As illustrated in FIG. 2, coupling members 46 and 47 for coupling the proximal end side of the catheter shaft 11 to the handle body 121 are arranged on the distal end side of the rotation operation portion 122.

The path defining member 42 is a member that defines a non-rotational path 420 through which the deformation wire 60 passes. The non-rotational path 420 is a linear path along the axial direction (Z-axis direction) of the handle 12 as illustrated in FIG. 3, and is a path that passes through the rotation axis Yr as illustrated in FIG. 2. The rotating member 43 is a member that rotates within the Z-X plane together with the rotating plate 41. As illustrated in FIG. 2, the ring-shaped member 44a is arranged between the rotating plate 41 and the path defining member 42, and the ring-shaped member 44b is arranged between the rotating plate 41 and the rotating member 43. As illustrated in FIG. 2, the connecting member 45 is a member for connecting the rotating plate 41, the path defining member 42, the rotating member 43, and the ring-shaped members 44a and 44b so as to be sandwiched between the handle members 121a and 121b together with the lock mechanism 40.

As illustrated in FIG. 3, in the handle body 121 (handle member 121b), a path through which the irrigation liquid L flows and a path through which the lead wire 50 passes are arranged separately from each other. Specifically, the paths of the liquid L and the lead wire 50 are arranged separately from each other so as to be opposite to each other with the slide mechanism 123 (such as a knob 123a described later) interposed therebetween.

Here, as illustrated in FIGS. 1A and 1B and FIGS. 3 and 4, a slide mechanism 123 slidable along the axial direction (Z-axis direction) in the handle body 121 (see arrow d3 in FIGS. 1A and 1B) is provided in the handle 12 (handle body 121). The slide mechanism 123 is a portion that is operated (slide operation) by the operator during a deformation operation in which the shape of the near-distal end structure 6 is changed between the non-deployed shape (petal shape) and the deployed shape (basket shape) which have been described above. As illustrated in FIGS. 1A and 1B and FIG. 3, such a slide action of the slide mechanism 123 is performed along a rail (opening portion) in the Z-axis direction formed on the handle body 121 (handle members 121a and 121b).

As illustrated in FIGS. 1A and 1B and FIGS. 3 and 4, the slide mechanism 123 is provided with a pair of knobs 123a and 123b, which are portions actually operated during the deformation operation. As illustrated in FIG. 4, these knobs 123a and 123b are arranged on both the front surface side and the back surface side of the handle body 121 so as to face each other along the Y-axis direction. As a result, when the slide mechanism 123 slides along the axial direction (Z-axis direction), the knobs 123a and 123b also slide on both the front surface side and the back surface side of the handle body 121, respectively.

Further, the slide mechanism 123 described above can be set to any slide position along the axial direction (Z-axis direction) on the handle body 121. Thus, the shape of the near-distal end structure 6 during the deformation operation can be set to any intermediate shape between the non-deployed shape (petal shape) and the deployed shape (basket shape) according to the slide position of the slide mechanism 123.

Here, the distal end side of the deformation wire 60 used during such a deformation operation is fixed to the near-distal end structure 6 (near the distal end tip 110). On the other hand, as illustrated in FIG. 4, the proximal end side of the deformation wire 60 is fixed by the slide mechanism 123 in the handle body 121 in a state of being inserted into a rigid tube 125a (lead pipe). Specifically, the rigid tube 125a is fixed to a sleeve 127a on the handle body 121 by a screw 127b formed at the knob 123a, whereby the proximal end side of the deformation wire 60 is fixed. Such a rigid tube 125a is a highly rigid tube that is difficult to bend and extends along the axial direction (Z-axis direction) of the handle body 121.

As illustrated in FIGS. 4 and 5, in the handle body 121, the distal end-side region of the rigid tube 125a is configured to be insertable into a protective tube 125b (protective pipe). Specifically, in the example state illustrated in FIGS. 4 and 5, the distal end-side region of the rigid tube 125a is inserted into the protective tube 125b. The irrigation liquid L flows into the gap between the protective tube 125b and the rigid tube 125a toward the catheter shaft 11 side (the near-distal end structure 6). Further, in the handle body 121, as illustrated in FIG. 4, a liquid stop member 126 is provided on the proximal end side of the inflow position of the liquid L with respect to the gap. The liquid stop member 126 is a member for suppressing leakage of the irrigation liquid L (leakage from the gap to the proximal end side). The liquid stop member 126 described above is formed using an O-ring, for example. Note that the liquid stop member 126 may also have a function of adjusting a load of the slide mechanism 123, for example.

Further, as illustrated in FIG. 5, the proximal end side of the protective tube 125b is connected to an irrigation liquid tube 125c via a relay member 128 inside the handle body 121.

Inside the irrigation liquid tube 125c, as illustrated in FIG. 5, the irrigation liquid L flows toward the distal end side, and the deformation wire 60 is inserted therein. Note that the irrigation liquid tube 125c described above is connected to the catheter shaft 11.

### C. Action and Functions/Effects

Next, the action and functions/effects of the electrode catheter 1 of the present embodiment will be described in detail while comparing with a comparative example.

### C-1. Deflection Action of Distal End Flexible Portion 11A by Rotation Operation

First, in the electrode catheter 1, the shape of the catheter shaft 11 near the distal end (distal end flexible portion 11A) changes in two directions according to the rotation operation of the rotating plate 41 by the operator. That is, in measuring the electric potential near an affected area in the body or cauterizing the affected area as described above, the action of deflecting the distal end flexible portion 11A in two directions (the deflection action in two directions) is performed in response to the rotation operation described above.

Specifically, for example, when the operator grips the handle 12 (handle body 121) with one hand and operates the knob 41a with the fingers of the one hand to rotate the rotating plate 41 in the direction of arrow d1a (clockwise) in FIG. 1A, the following is achieved. That is, one deflection wire of the pair of deflection wires is pulled toward the proximal end side inside the catheter shaft 11. Then, the distal end flexible portion 11A of the catheter shaft 11 is curved (bent) along the direction indicated by arrow d2a in FIG. 1A.

Further, for example, when the operator operates the knob 41b to rotate the rotating plate 41 in the direction of arrow d1b (counterclockwise) in FIG. 1A, the following is achieved. That is, the other deflection wire of the pair of deflection wires is pulled toward the proximal end side inside the catheter shaft 11. Then, the distal end flexible portion 11A of the catheter shaft 11 is curved along the direction indicated by arrow d2b in FIG. 1A.

In this manner, the operator can perform a (swing) deflection action in two directions in the catheter shaft 11 by rotating the rotating plate 41. By rotating the handle body 121 about its axis (within the X-Y plane), the bending direction (deflection direction) of the distal end flexible portion 11A of the catheter shaft 11 can be freely set in a state where the catheter shaft 11 is inserted into the patient's body, for example. In this manner, the electrode catheter 1 is provided with a deflection mechanism for deflecting the distal end flexible portion 11A in two directions, so that the catheter shaft 11 can be inserted into the patient's body while changing the shape near its distal end (distal end flexible portion 11A).

As described above, the above-described potential measurement and cauterization (ablation) are performed at the distal end flexible portion 11A (the near-distal end structure 6 including the plurality of electrodes 111).

Further, in the present embodiment, the above-described irrigation liquid L is supplied to the electrode catheter 1 during the ablation. Specifically, for example, as illustrated in FIG. 1A, the liquid L is supplied into the handle body 121 from a side surface (liquid inlet) on the proximal end side of the handle body 121. Then, for example, as illustrated in FIG. 1A, the liquid L flows out (is ejected) to the outside from the vicinity of the distal end of the electrode catheter 1 (the vicinity of the above-described branch point in the near-distal end structure 6). This avoids that damage is caused by excessive increase in the temperature of the procedure part during ablation and that a thrombus sticks to the procedure part (blood retention is improved).

### C-2. Deformation Action of Near-Distal End Structure 6 by Deformation Operation

Next, referring to FIGS. 6A to 6C and FIGS. 7A to 7C, the deformation action of the near-distal end structure 6 of the catheter shaft 11 by the deformation operation on the slide mechanism 123 will be described in detail.

FIGS. 6A to 6C schematically illustrate an example of a deformed state (the state of the petal shape as an example of the non-deployed shape) near the distal end of the catheter shaft 11 (the near-distal end structure 6). FIGS. 7A to 7C schematically illustrate an example of another deformed state (the state of the basket shape as an example of the deployed shape) near the distal end of the catheter shaft 11 (the near-distal end structure 6). Note that the deployed shape (basket shape) illustrated in FIGS. 7A to 7C is merely an example, and may be, for example, a shape slightly deflated (distorted) from the shape illustrated in FIGS. 7A to 7C.

First, as indicated by arrow d3a in FIG. 6A, for example, when the slide mechanism 123 slides toward the proximal end side of the handle body 121 by a deformation operation of the slide mechanism 123 by the operator, the following is achieved. That is, as described above, the proximal end side of the deformation wire 60 is fixed by the slide mechanism 123, In this case, for example, as indicated by arrow d4a in FIGS. 6A to 6C, as the slide mechanism 123 slides toward the proximal end side, the deformation wire 60 is also pulled toward the proximal end side. Then, as described above, since the distal end side of the deformation wire 60 is fixed to the near-distal end structure 6 (near the distal end tip 110), for example, as illustrated in FIGS. 6B and 6C, the distal end tip 110 is pulled toward the proximal end side, and a shape in which the branch structures 61a to 61e are contracted toward the proximal end side is obtained. That is, the near-distal end structure 6 has the non-deployed shape (in this example, a shape substantially flattened in the X-Y plane). Specifically, in this example, as illustrated in FIG. 6B, the near-distal end structure 6 has the petal shape formed by the branch structures 61a to 61e.

On the other hand, as indicated by arrow d3b in FIG. 7A, for example, when the slide mechanism 123 slides toward the distal end side of the handle body 121 by the deformation operation on the slide mechanism 123 by the operator, the following is achieved. That is, in this case, as indicated by arrow d4b in FIGS. 7A to 7C, for example, as the slide mechanism 123 slides toward the distal end side, the deformation wire 60 is also pushed toward the distal end side. Then, for example, as illustrated in FIGS. 7B and 7C, the distal end tip 110 is pushed toward the distal end side, and a shape in which the branch structures 61a to 61e are deployed toward the distal end side is obtained. That is, the near-distal end structure 6 has the deployed shape (a shape in which the near-distal end structure 6 is deployed toward the distal end side along the Z-axis direction). Specifically, in this example, as illustrated in FIG. 7B, the near-distal end structure 6 has the basket shape formed by the respective branch structures 61a to 61e.

In this manner, the near-distal end structure 6 is deformed according to the deformation operation on the slide mechanism 123.

### C-3. Comparative Example

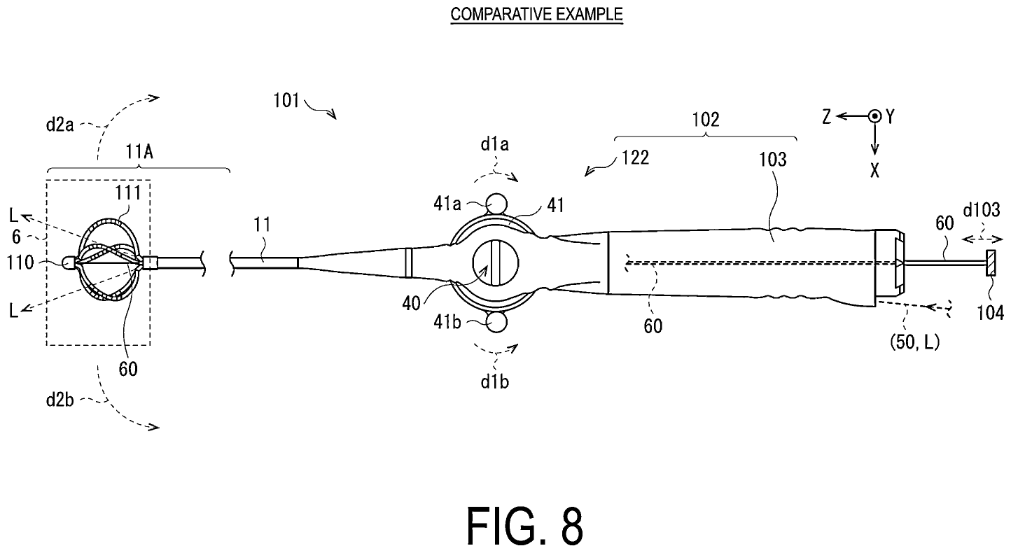

Here, FIG. 8 is a schematic plan view (Z-X plan view) illustrating a schematic configuration of a catheter (electrode catheter 101) according to a comparative example.

The electrode catheter 101 of this comparative example includes a catheter shaft 11 having a near-distal end structure 6, and a handle 102 having a handle body 103 and a rotation operation portion 122. In other words, the electrode catheter 101 of this comparative example has the handle 102 and the handle body 103 instead of the handle 12 and the handle body 121 in the electrode catheter 1 (see FIGS. 1A and 1B) of the present embodiment.

Specifically, as illustrated in FIG. 8, in this handle body 103, a push-in operation portion 104 is provided instead of the slide mechanism 123 in the embodiment. The proximal end side of the deformation wire 60 taken out from the proximal end of the handle body 103 is attached to the push-in operation portion 104. When the operator operates the push-in operation portion 104 along the direction of arrow d103 (the Z-axis direction: the extension direction of the deformation wire 60), an operation of pushing the deformation wire 60 into the handle body 121 is performed. As a result, the near-distal end structure 6 is deformed in the same manner as in the present embodiment. That is, in this comparative example, the operation in the direction of arrow d103 with respect to the push-in operation portion 104 corresponds to a deformation operation for deforming the near-distal end structure 6.

In the handle body 103 of this comparative example, unlike the handle body 121 of the present embodiment illustrated in FIGS. 1A and 1B, the irrigation liquid L is introduced from the side surface of the handle body 103 on the proximal end side, and the lead wire 50 is also pulled out. That is, since the deformation wire 60 is pulled out from the proximal end of the handle body 103, unlike the present embodiment, the lead wire 50 is pulled out from the side surface rather than from the proximal end of the handle body 103.

In such a comparative example, in a state where the operator grips the handle body 103 with one hand, the above-described operation (deformation operation) on the push-in operation portion 104 is performed using the other hand of the operator. Thus, since an operation by both hands of the operator is required during the deformation operation using the push-in operation portion 104, the deformation operation for deforming the near-distal end structure 6 becomes complicated (it is difficult to easily perform the deformation operation).

In this manner, convenience of using the electrode catheter 101 of the comparative example is impaired.

### C-4. Present Embodiment

On the other hand, in the electrode catheter 1 of the present embodiment, the following functions/effects are obtained with the configuration described above.

First, in the electrode catheter 1 of the present embodiment, the handle 12 is provided with the slide mechanism 123 that is slidable along the axial direction (Z-axis direction) of the handle body 121. As a result, the deformation operation in which the shape of the near-distal end structure 6 including the plurality of electrodes 111 is changed between the non-deployed shape (the petal shape) and the deployed shape (basket shape) is performed as follows. That is, in a state where the operator holds the handle body 121 with one hand, the operator can perform the deformation operation to the slide mechanism 123 with the one hand (the same hand). That is, for example, as in the comparative example described above, an operation by both hands of the operator as in the case of an operation of pushing the deformation wire 60 against the handle body 121 using the other hand is not required, and the deformation operation can be easily performed using only one hand of the operator. As a result, the convenience of using the electrode catheter I can be improved in the present embodiment.

Further, since the deformation operation is performed using the slide mechanism 123, the following effects can be obtained, for example. That is, for example, with the handle 12 placed on a predetermined table, it is possible to easily perform the above-described rotation operation with one hand while performing the deformation operation with the other hand. Further, unlike the handle body 103 in the comparative example, the lead wire 50 can be easily pulled out from the proximal end of the handle body 121 in a state of being separated from the inflow path of the irrigation liquid L.

Further, in the present embodiment, since the shape of the near-distal end structure 6 can be set to any intermediate shape between the non-deployed shape and the deployed shape according to the slide position of the slide mechanism 123 in the handle body 121, the convenience can be further improved.

In addition, in the present embodiment, the distal end side of the deformation wire 60 is fixed to the near-distal end structure 6, and the proximal end side of the deformation wire 60 is fixed by the slide mechanism 123 in a state of being inserted into the rigid tube 125a in the handle body 121, the following is achieved. That is, since the proximal end side of the deformation wire 60 is inserted into the rigid tube 125a inside the handle body 121, the deformation wire 60 is avoided from being bent inside the handle body 121 when the deformation operation is performed. As a result, the deformation operation can be performed smoothly, and the convenience can be further improved.

In addition, in the present embodiment, the distal end-side region of the rigid tube 125a in the handle body 121 is configured to be insertable into the protective tube 125b, and configured so that the irrigation liquid L flows into the gap between the protective tube 125b and the rigid tube 125a toward the near-distal end structure 6. The liquid stop member 126 is provided in the handle body 121 on the proximal end side of the inflow position of the liquid L with respect to the gap. As a result, the irrigation liquid L can be supplied toward the near-distal end structure 6 using the gap between the protective tube 125b and the rigid tube 125a, and the leakage of the irrigation liquid L can be suppressed by providing the liquid stop member 126. As a result, the size of the handle body 121 can be reduced, and the reliability of the handle 12 as a product can be improved.

Further, in the present embodiment, since the rotation operation portion 122, which is rotated during the deflection action, includes the rotating plate 41 and the path defining member 42 formed using a member different from a member of the rotating plate 141, the path defining member 42 determining the non-rotational path 420 through which the deformation wire 60 passes, the following is achieved. That is, since the rotating plate 41 and the path defining member 42 are formed using different members, the non-rotational path 420 of the deformation wire 60 is maintained even during the rotation operation. As a result, the rotation operation and the deformation operation can be easily performed with a simple structure, thereby further improving the convenience.

In addition, in the present embodiment, since the knobs 123a and 123b, which are portions to be operated during the deformation operation, are provided in the slide mechanism 123, and these knobs 123a and 123b are arranged on both the front surface side and the back surface side of the handle body 121, the following is achieved. That is, when the slide mechanism 123 slides along the axial direction (Z-axis direction). the knobs 123a and 123b slide on both the front surface side and the back surface side of the handle body 121. Thus, during the deformation operation, the rotational movements from both knobs 123a and 123b in the intermediate direction cancel each other out. This avoids the possibility of loss of the operating load during the deformation operation, and as a result, the deformation operation can be performed smoothly, and the convenience can be further improved.

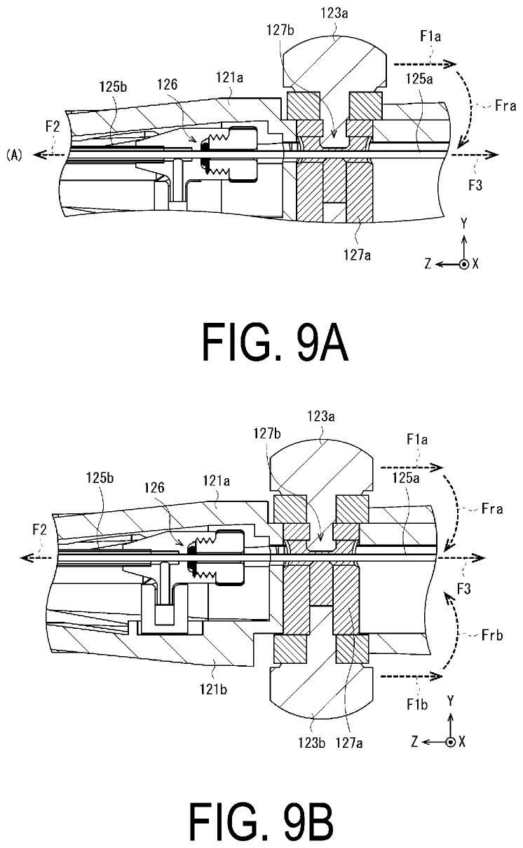

Specifically, for example, as illustrated in FIG. 9A, when only one (knob 123a) of the above-described knobs 123a and 123b is provided in the slide mechanism 123, there is a possibility that the operating load during the deformation operation is impaired as follows. That is, first, when an operating load F1a from the knob 123a to the proximal end side is generated during the deformation operation using the knob 123a, the near-distal end structure 6 tries to return to the original shape (the basket shape), and a restoring force F2 toward the distal end side is generated in the deformation wire 60. Then, when the operating load F1a and the restoring force F2 try to balance each other, these directions try to align on a straight line (along the extension direction of the deformation wire 60). Thus, the direction of the operating load F1a is aligned with the direction of arrow F3, and a rotational movement is generated by a rotational force Fra. When such a rotational movement occurs, an excessive interference occurs between the operating load F1a and the restoring force F2, and as a result, the operating load during the deformation operation may be impaired.

On the other hand, for example, as illustrated in FIG. 9B (and FIG. 4 described above), when both of the knobs 123a and 123b are provided in the slide mechanism 123, the following is achieved. That is, first, during the deformation operation using both of the knobs 123a and 123b, operating loads F1a and F1b from the knobs 123a and 123b to the proximal end side are generated, respectively. Thus, when the operating loads F1a and F1b and the restoring force F2 try to balance each other, the rotational forces Fra and Frb (mutual rotational movements) in the direction of arrow F3 due to the operating loads F1a and F1b cancel each other out. As a result, the case of FIG. 9B (the present embodiment), unlike the case of FIG. 9A, can avoid the possibility that the operating load during the deformation operation is damaged.

### 2. Modified Examples

The disclosure has been described with reference to the embodiment, but the disclosure is not limited to the embodiment, and various modifications can be made.

For example, the shape, arrangement position, size, number, material, and the like of components described in the above-described embodiment are not limited, and other shapes, arrangement positions, sizes, numbers, materials, and the like may be used.

Specifically, for example, in the above-described embodiment, the configuration of the catheter shaft 11 has been described in specific as an example, but it is not always necessary to include all of the members, and other members may be further included. Specifically, for example, a leaf spring that can be deformed in the deflection direction may be provided as a swing member inside the catheter shaft 11. Further, for example, the arrangement, shape, and number of (one or a plurality of) the respective electrodes 111 near the distal end of the catheter shaft 11 (in the near-distal end structure 6) are not limited to those described in the above-described embodiment. In addition, the shape of the near-distal end structure 6 (the non-deployed shape and the deployed shape) is not limited to the shapes described in the embodiments (the petal shape, the basket shape, and the like as an example of the flat shape) but may be other non-deployed shapes or other deployed shapes. Furthermore, the configuration of the near-distal end structure 6 itself (for example, the arrangement, shape, and number of the branch points, the joining points, and the plurality of branch structures described above) are not limited to the configuration described in the above-described embodiment, and may be other configurations.

Further, in the embodiment, the configurations of the handle 12 (the handle body 121, the rotation operation portion 122, the slide mechanism 123, and the like) have been described specifically. However, it is not always necessary to include all the members, and other members may be further included. Specifically, for example, the type of shape when the near-distal end structure 6 is deformed is not limited to the case where the shape can be set to any intermediate shape as described in the embodiment, but may be set to another case. That is, for example, the shape may be set to only a plurality of types of preset intermediate shapes instead of any intermediate shape, or for example, the shape may be set to only two types of shapes of the non-deployed shape and the deployed shape (cannot be set to the intermediate shape). In the embodiment, a case where the slide mechanism 123 is provided with the pair of knobs 123a and 123b has been described as an example. However, for example, only one of the knobs 123a and 123b may be provided. In addition, in the embodiment, a case where the rotating plate 41 and the path defining member 42 are formed of different members has been described as an example. However, the disclosure is not limited to this case, and other configurations may be used. In addition, the configurations of the rigid tube 125a, the protective tube 125b, the liquid stop member 126, and the like are not limited to the configurations described in the embodiment, and other configurations may be used.

In addition, the shape near the distal end of the catheter shaft 11 is not limited to that described in the embodiment. Specifically, in the embodiment, the electrode catheter 1 of a type (bi-direction type) in which the shape near the distal end of the catheter shaft 11 changes in two directions according to the operation of the rotating plate 41 has been described as an example. However, the type of electrode catheter 1 is not limited to this. That is, for example, the electrode catheter may be of a type (single-direction type) in which the shape near the distal end of the catheter shaft 11 changes in one direction according to the operation of the rotating plate 41. In this case, only one operation wire is provided.

In addition, in the embodiment, the electrode catheter 1 that ejects the irrigation liquid L to the outside (having an irrigation mechanism) has been described as an example. However, the disclosure is not limited to this example, and may be applied to, for example, an electrode catheter that does not have such an irrigation mechanism. Further, in the embodiment and the like, the electrode catheter 1 that performs the above-described potential measurement and cauterization (ablation) has been described as an example. However, the disclosure is not limited to this example, and may be applied to, for example, an electrode catheter used for other applications.

While preferred embodiments of the disclosure have been described above, it is to be understood that variations and modifications will be apparent to those skilled in the art without departing from the scope and spirit of the disclosure. The scope of the disclosure, therefore, is to be determined solely by the following claims.

1. A catheter comprising:
a catheter shaft extending along an axial direction and including a near-distal end structure including a plurality of electrodes; and
a handle mounted on a proximal end side of the catheter shaft, wherein
the handle includes:
a handle body extending along the axial direction; and
a slide mechanism configured to be slidable along the axial direction in the handle body and operated during a deformation operation in which a shape of the near-distal end structure is changed between a first shape and a second shape,
the first shape is a non-deployed shape in which the near-distal end structure is not deployed along the axial direction, and
the second shape is a deployed shape in which the near-distal end structure is deployed from the non-deployed shape along the axial direction.

2. The catheter according to claim 1, wherein the near-distal end structure is settable to any intermediate shape between the non-deployed shape and the deployed shape according to a slide position of the slide mechanism in the handle body.

3. The catheter according to claim 1, wherein
a distal end side of a deformation wire used in the deformation operation is fixed to the near-distal end structure, and
a proximal end side of the deformation wire is fixed by the slide mechanism in a state of being inserted into a rigid tube in the handle body.

4. The catheter according to claim 3, wherein
the near-distal end structure is configured such that an irrigation liquid is ejected to an outside,
a distal end-side region of the rigid tube in the handle body is configured to be insertable into a protective tube, such that the liquid flows into a gap between the protective tube and the rigid tube toward the near-distal end structure, and
a liquid stop member configured to suppress leakage of the liquid is provided in the handle body on a proximal end side of an inflow position of the liquid with respect to the gap.

5. The catheter according to claim 1, wherein
the handle further includes a rotation operation portion that is rotated during a deflection action of deflecting a portion near a distal end of the catheter shaft, and
the rotation operation portion includes:
a rotating plate configured to be rotatable about a rotation axis perpendicular to the axial direction with respect to the handle body, the rotating plate being a portion operated when the rotation operation portion is rotated; and
a path defining member formed using a member different from a member of the rotating plate, the path defining member being configured to define a non-rotational path through which the deformation wire used in the deformation operation passes.

6. The catheter according to claim 1, wherein
the slide mechanism includes a knob which is a portion operated during the deformation operation, and
the knob is disposed on both a front surface side and a back surface side of the handle body.

7. The catheter according to claim 1, wherein
the near-distal end structure includes:
a branch point of the catheter shaft;
a joining point located near a most distal end of the catheter shaft; and
a plurality of branch structures respectively including the electrode, the plurality of branch structures individually connecting the branch point and the joining point in a curved shape,
the non-deployed shape is a petal shape formed by the plurality of branch structures, and
the deployed shape is a shape in which the petal shape is deployed along the axial direction.
